(19) United States
(12) Patent Application Publication
(10) Pub. No.: US 2020/0246166 A1
(43) Pub. Date: Aug. 6, 2020

(54) REMOVABLE APPARATUS FOR TREATMENT OF VEIN CONSTRICTION

(71) Applicant: Agents of Healing, LLC, Perrysburg, OH (US)

(72) Inventors: Anthony Semaan, Napoleon, OH (US); Don Smucker, Perrysburg, OH (US)

(73) Assignee: Agents of Healing, LLC, Perrysburg, OH (US)

(21) Appl. No.: 16/783,854

(22) Filed: Feb. 6, 2020

Related U.S. Application Data
(60) Provisional application No. 62/801,951, filed on Feb. 6, 2019.

(51) Int. Cl. A61F 2/92; A61F 2/95

(52) U.S. Cl. CPC A61F 2/92; A61F 2002/823; A61F 2/95

(57) ABSTRACT

A retrievable stent and retrieval catheter for biological usage.

### BACKGROUND OF THE INVENTION

There are central veins in chest, abdomen and pelvis “Root Veins,” and peripheral veins in arms and legs. Peripheral veins in extremities can be Deep Veins inside muscle or Superficial Veins in soft tissue between muscle and skin surface. Extremities veins both deep and superficial contain flaps, also known as valves. When muscles contract, the valves open and allow blood to move through the veins towards the heart. When muscles relax, the valves close to prevent blood back flow away from the heart, therefore keeping blood flowing in one direction through the veins. If veins valves become damaged, as a result of vein disease such as dilatation or clot, the valves may not close completely allowing blood to leak backward, leading to vein congestion and eventually leak out of the vein wall to surrounding body tissue. Chronic vein disease is common in adults 41-60 years to very common in seniors above 60 years, with more than 200,000 new cases per year in the United States alone.

Venous disease is known to result in blood clots, deep vein thrombosis (DVT), superficial venous thrombosis or phlebitis, chronic venous insufficiency, spider veins, varicose veins, and venous ulcers. The most common therapy used in vein clinics today are; compression stockings, sclerotherapy medications, vein ablation and even surgery (ligation, vein stripping). Further the inventors believe that current vein therapies treat the tip of the iceberg; the symptoms rather than the underlying root cause. The inventors also believe chronic vein disease are much more common than currently known, starting earlier than currently thought and likely affecting the entire body. Kids, teens and young adults may notice, but dismiss or get used to, subtle symptoms such as leg heaviness, aching, and restless legs. In fact, these symptoms are likely due to early and subtle vein circulation disease caused by root central veins leading to early venous congestion mainly in legs, veins, abdomen and pelvis. Overtime, small veins erupt causing spider veins (can be visible or internal). Eventually the feeding vessels to the spider veins will dilate and bulge, resulting in visible varicosities on skin surface. Simply the body overtime creates and/or enlarges veins to contain blood that is congested and not able to flow back swiftly to the heart. With time, these dilated, bulging vessels begin to leak into the surrounding soft tissue, causing edema, swelling, skin inflammation, redness, brown discoloration and hyper pigmentation. If left untreated, the skin will eventually deteriorate, and an ulceration will form.

The adverse effects internally on the body are not well understood, but the inventors believe this vein disease concept may contribute to other body chronic illnesses affecting the entire population. Once the veins become dilated or varicosed, deoxygenated blood containing toxins from the body's organs can begin to pool and leak out of the dilated bulging veins. This pooling, deoxygenated, toxin laden blood is known to cause breakdown of tissue and ulceration in the legs, however, it is hypothesized that the same process would occur in other locations even at or around multiple body organs. And therefore, the inventor proposes a method and apparatus for treating this underlying cause and the chronic illnesses that result from pooling of the deoxygenated, toxin laden blood.

### SUMMARY OF THE INVENTION

The inventors proposed a new theory for chronic disease etiology and treatment. The etiology being the body central root veins are either congenitally compromised, narrowed or compressed or slowly impaired during growth and aging, gradually impacting body organs and leading to chronic disease/organs dysfunction. The inventors believe it is the underlying cause of most if not all chronic illnesses. The central root veins are iliac veins, inferior vena cava (IVC), superior vena cava (SVC), subclavian and brachiocephalic veins.

This theory is new and not well understood. The central root veins are a major center of the circulation system, directly impact the entirety of vascular system, and therefore the entire body. It is very likely the under lying root cause for many chronic health conditions, By understanding this and treating early, this novel approach can lead to major advancement in chronic disease prevention, treatment, improved quality of health and potentially reduce treatment costs that currently treat the tip of the iceberg, rather than the underlying root cause.

The inventors have further proposed a treatment method and apparatus for the treatment of underlying root cause of vein disease and other chronic illnesses resulting from central vein disease, including but not limited to, chest pains, hypertension and hardening of arteries, blood clots in lungs known as deep venous thrombosis DVT/PE, chronic headaches, migraines, restlessness leg syndrome, poor concentration, gallbladder dysfunction, irritable bowel syndrome, inflammatory bowel disease, undiagnosed reason for abdominal pain, constipation, lower back pain, fibromyalgia, endometriosis, chronic pelvic pain, dementia (sundowning), ADHD, autism, anxiety, depression, chronic narcotic addiction, asthma, intra uterine growth restriction, pregnancy related leg swelling and varicose veins, postpartum depression, placenta congestion, pelvic bleeding during pregnancy, miscarriages, eclampsia, and preeclampsia, Musculoskeletal disease such as chronic low back pain, failed back syndrome, disc herniation, osteoarthritis, sacroiliitis, disc deterioration not related to injury, and sciatica.

The vast majority of vein disease treatments today treat the symptom rather than the cause of the vein disease. The inventors hypothesize that the human venous system is akin to a cities road system. Wherein the central veins are akin to highways. As the highway becomes clogged, compressed and narrowed traffic seeks alternate routes. Similarly, if blood is not flowing properly through a vein due to constricted flow, the blood will seek alternative paths resulting inside veins, varicosities and ultimately blood pooling outside of the veins. Blood within the vein system has been deoxygenated and contains toxins from various organs in the human body. If this toxin laden, deoxygenated blood pools around tissue it causes the cells to break down, ultimately resulting in ulcers in the skin or internal organs dysfunction or chronic disease.

The superior vena cava is a large vein that brings blood from the head and arms to the heart, while the inferior vena cava brings blood from the abdomen, pelvis and legs into the heart. The aorta also runs from the heart to the lower extremities. This major vein and major artery converge and overlap in the abdomen (around the L4, L5 vertebra) where the aorta and its bifurcation pass over, or lays on top of, the inferior vena cava bifurcation. The blood pressure within the aorta in a normal human is typically between 105 and 120 mmHg but can be as high as over 240 mmHg in patients with hypertension. Conversely, vein pressure is usually around 5-20 mmHg in a normal adult patient but can be as high as 40 mmHg. As such, the blood pressure in the aorta is of a magnitude greater than the pressure in the inferior vena cava. The inferior vena cava is thus placed between the human spine and the aorta, both being significantly firmer than the inferior vena cava.

This placement of the inferior vena cava bifurcation between the spine and the aorta and iliac arteries can restrict the flow of blood through the inferior vena cava. As the heart beats and forces blood through the aorta at a significantly higher pressure, the aorta presses against and compresses the inferior vena cava. Typically, this compression occurs in the left iliac vein. However, it is not uncommon for the compression to occur in the right iliac vein, or even in both. This compression restricts the blood flow back to the heart and causes blood to stagnate and seep through the vein, pooling at low points in the body. One such low point is at the L4L5-S1 vertebra, which is also the most common area of chronic back pain. Absent an acute injury, the presence of back pain at the L4L5-S1 vertebra may be attributable to this pooling of toxin laden, deoxygenated blood, as the deoxygenated blood will affect the vertebra in the same way it affects the lower legs, ultimately resulting in skin ulceration if untreated.

Sedentary and high fat diets may exacerbate this problem. As humans gain weight more pressure is put on the abdomen area which may increase the compression seen in the iliac vein.

It is possible for a treating physician to perform an angiogram of the vein using contrast dye infused in the vein, along with an x-ray to detect the contrast. However, this technique even though useful to map the pattern of venous flow can mask vein restrictions and compression, as the injection high pressure itself can actually expand the compressed vein. Therefore, the preferred method would be an intravascular ultrasound device which can calculate the actual degree of compression/restriction by precisely measuring the internal diameter and surface area of the compressed vessel. A treating physician can perform both tests in a minimally invasive technique while the patient is under mild to moderate anesthesia. If a compression of the central vein is present, the condition can be treated with a stent.

Placement of a stent at the compression site can restore normal blood flow through the inferior vena cava reversing or greatly reducing the effects of venous disease and other chronic illnesses associated with the pooling of the toxin laden, deoxygenated blood. However, the current available stents manufactured by numerous companies are not ideal. One such currently available stent, known as a “wall stent,” manufactured by Boston Scientific. All available stents including the wall stent are not optimal because it will collapse when pressure is applied to it (the blood pressure from the aorta would be sufficient to collapse the wall stent especially at the stent ends), therefore it will not reduce or eliminate the compression. Further, the wall stent is sharp on both ends which create problems in fragile thin veins. It is limited in length, often requiring multiple stents to be coupled together while overlapping which leads to increase metal content at area of overlap and increase footprint. The wall stent, for example, is not specifically designed for use in veins, the sharp edges of the stent can cause damages to the vein walls which can increase the chance of blood clot. Further, none of the current stents, including the wall stent is not retrievable nor is there any device known for retrieving stents after deployment. A retrievable stent would be desirable for treatment of the compression in the iliac vein. The stent could be placed in younger patients without complicating future issues, such as pregnancy, as the stent could easily be removed.

Therefore, there is a need for a retrievable stent suitable for treatment to correct the compression in the iliac vein.

Recently in mid-2019 additional stents were approved for use in veins including VICI stent by Boston Scientific and Venovo stent by Bard company, both of these stents were made of larger content of metal surface and do not conform well the natural anatomy of the veins, they can stretch the vein more than what it should be naturally, and the increased footprint increases risk of scaring and blood clotting.

### BRIEF DESCRIPTIONS OF THE DRAWINGS

FIG. 1 is a side view of the spring stent without an extraction point.

FIG. 2 is a side view of the spring stent with an eyelet extraction point.

FIG. 3 is a side view of the spring stent with a T shaped anchor extraction point.

FIG. 4 is a side view of the insertion/retrieval catheter.

FIG. 5 is a side view of the insertion/retrieval catheter with a spring stent loaded onto the guide wire.

FIG. 6 is an end side view of the spring stent with an eyelet extraction point.

FIG. 7 is an end side view of the spring stent with a T shaped anchor extraction point.

### DETAILED DESCRIPTION OF THE INVENTION

In the preferred embodiment, a retrievable flexible stent specifically designed for veins or arteries would be used. This new stent should be more rigid than a wall stent but still maintain some flexibility for placement and retrieval. The vein stent could be coated in a hydrogel or other anti-drug material. The vein stent would have smooth edges and in the preferred embodiment would be in a continuous loop without wires crossing over itself or a mesh, i.e. the shape of a spring. This vein stent or retrievable flexible spring stent would reduce the surface area of the stent by approximately 90% over that of a traditional wall stent. This reduction in surface area would result in less surface contact for blood platelets and other blood cells to attach during circulation through the stent. Thus, the spring stent would require less, or perhaps no blood thinner treatment after deployment. These features will reduce surface contact to reduce the risk of blood cells and platelets aggregation to the surface of the spring stent. A spiral slope of the spring stent will increase flow rate through the stent over commonly used stents today.

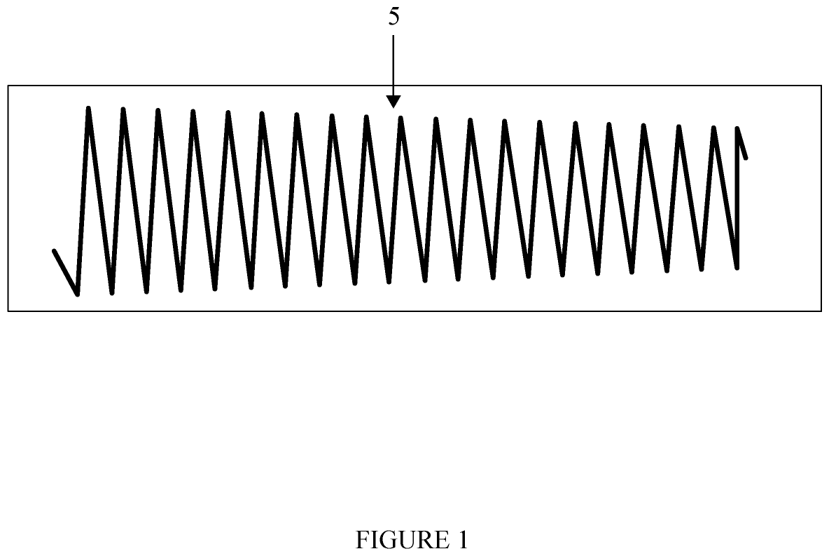

Referring now to FIGS. 1-3, in the preferred embodiment the wire of the spring stent 5 will taper from end to creating an oval shape in the wire. This oval shaping of the wire will help the spring stent conform to match the contour of the vessel wall. Further reducing the surface area of the spring stent.

In the preferred embodiment the spring stent 5 has a slightly taper shape to match the natural anatomy of the vessel being slightly smaller in diameter and surface area at the peripheral end (distal end).

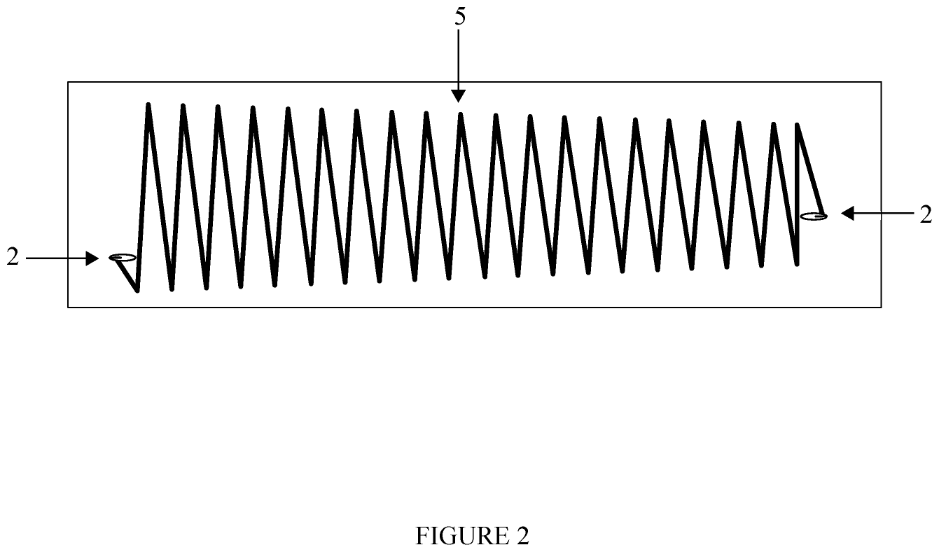

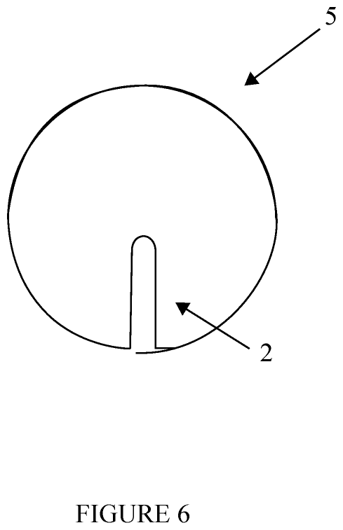

In the preferred embodiment the spring stent 5 has an extraction point on at least one end of the stent. In another embodiment, the extraction point is an eyelet 2 at one or both ends of the stent (FIGS. 2 and 6). The eyelet 2 allows an insertion/extraction tool 14 to attach to the spring stent 5 and partially collapse it. The eyelet 2 further allows the insertion/extraction tool 14 to tension the spring stent 5 for easier handling, placement and removal.

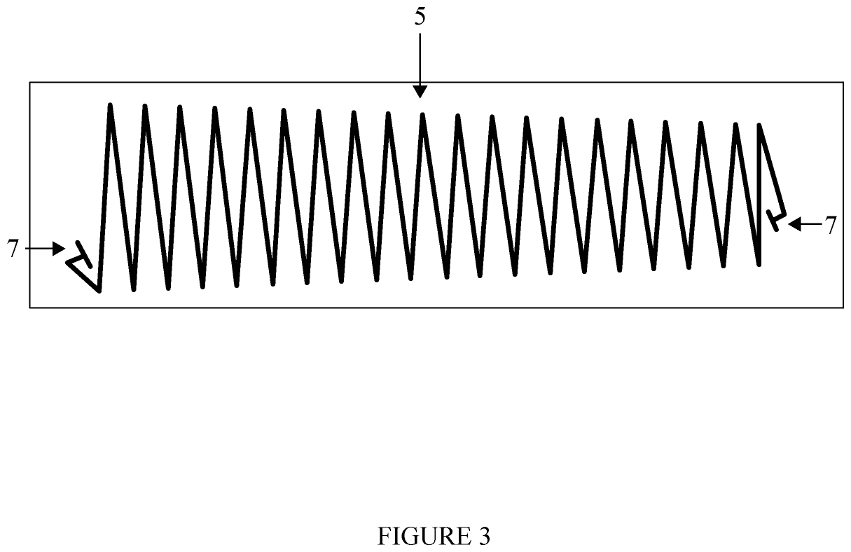

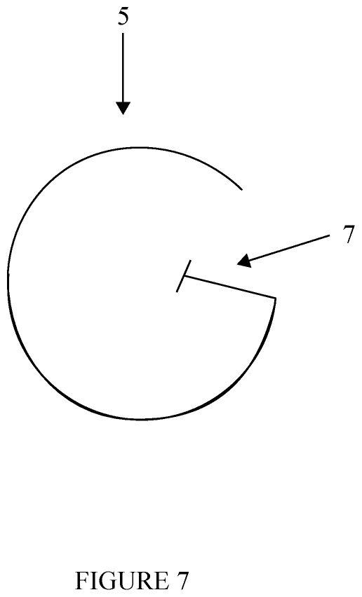

In yet another embodiment, the extraction point is a small anchor in the shape of a T 7 (FIGS. 3 and 7). Wherein, the spring wire end is bent inward, 30-35 degrees, to form the T shaped anchor. The long stem of the T is between 4-4.5 mm (30-35-degree angle from the spring end). The T top is about 2.5 mm (directed along the long axis of the spring/vessel) so circulating blood is “seeing” only a dot. This small T shaped anchor 7 maybe located on either or both ends of the spring stent 5 to allow capturing from either direction. While these dimensions are preferred, they are not integral to the design and any suitable dimension could be used. The small T shaped anchor would require a custom insertion/extraction 14 tool having a recessed area in the tip 18, capable of accepting the small T shaped anchor into itself.

Further, the retrievable spring stent 5 could terminate with other shapes as extraction points, provided that the extraction point can be attached to an insertion/retrieval catheter.

In another embodiment, micro fissures (not shown) are added to the distal end of the retrievable spring stent 5. The micro fissures provide adherence to the vessel wall and prevent risk of stent movement or migration but still allow for retrieval. The micro fissures prevent the spring stent 5 from migrating inside the vessel. The micro fissures could be located on both ends of the spring stent 5, or throughout the stent, but in the preferred embodiment micro fissures would be located on the distal end of the stent.

The retrievable spring stent 5 can be made from a memory shaped alloy suitable for insertion in the body, including but not limited to nitinol, gold, platinum, titanium, carbon fiber, polymers, high density plastic, UHMPE poly's, stainless steel, cobalt chrome alloy, nickel titanium allow, silver, tantalum, tungsten, aluminum oxide, zirconia, calcium phosphates, silicone, poly-ethylene, poly-vinyl chloride, polyurethanes, polylactides, and fiberglass.

In the preferred embodiment the spring stent 5 is made from Inconel. The Inconel provides wire strength and allows for x-ray visualization of the spring stent. Further the Inconel wire provides strength and recoil abilities, while providing a smooth surface area.

In another embodiment, a hydrophobic coating is added to the spring stent 5 to further decrease surface adherence. A hydrophobic coating over a composite spring stent, such as Inconel, makes the surface wet and less likely for blood cells to adhere. The coating also reduces adhesion to the vessel wall making retrieval easier. Further, an election charge can be added to the spring stent 5 to inhibit the vessel walls and blood cells from adhering to the stent. The electron charge would also reduce tissue scarring inside the vessel and helps resist attachment to the vessel walls.

The spring stent 5 can be produced in any diameter, but the most common diameters would be; 11-13.5 mm, 11-14 mm, 12-15 mm, 13-15.5 mm, 13-16 mm, 14-17 mm, 15-18 mm, 15-18.5 mm.

In another embodiment, the pitch (density) of the coils in the spring stent 5 is varied such that the coils are more dense at the ends of the spring stent and less dense in the middle of the stent. In the preferred embodiment the coils at the end of the spring stent 5 are spaced 1 mm apart, while the coils in the center of the stent are space 2.5 mm apart. This pitch variation yields more support where arteries cross over the vein, typically at the origin of the vein or point of bifurcation.

An insertion/retrieval catheter 14 will hook onto an extraction point 2 on one end of the spring stent 5 to deploy or retrieve the stent. When tension is attached to the extraction point 2 the spring stent 5 will begin to contract or wind so that it is smaller.

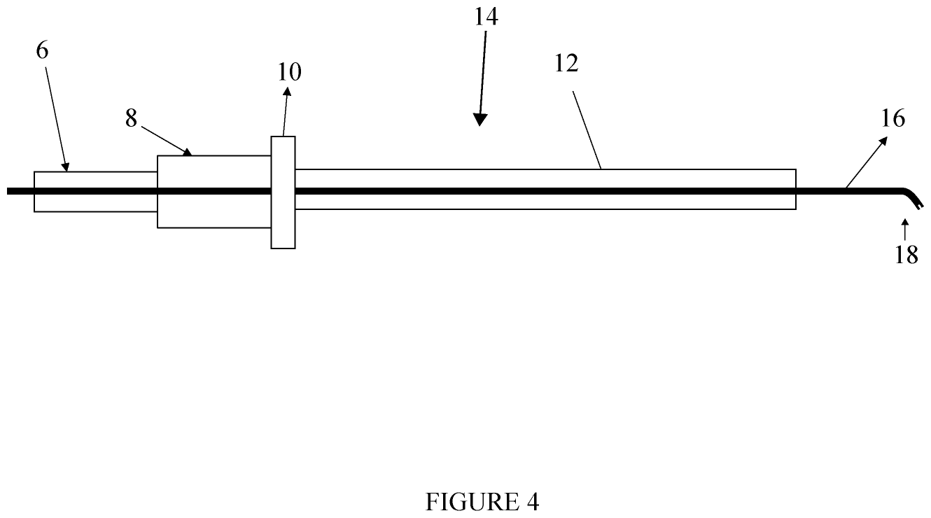

Referring now to FIGS. 4 and 4, in one embodiment, the insertion/retrieval catheter 14 comprises a compression collar 12 to contain the spring stent 5 during deployment or retrieval, a collar mount 8 and handle 6. The insertion/retrieval catheter 14 will further comprise a terminus end 18, slightly bent downward from the shaft of the guide wire 16. The terminus end 18 is milled to match the extraction point of the wall stent and form a locking cleat. The locking cleat engages the extraction point 2 of the spring stent 5 during insertion or retrieval. When acted on by the user, the locking cleat puts a force on the extraction point 2, causing the spring stent 5 to wind inward or outward as needed, thus shrinking or expanding the spring stent. This allows the spring stent 5 to be inserted into the vessel or retrieved from the vessel.

In another embodiment, the insertion/retrieval catheter 14 comprises a compression collar 12 to contain the spring stent 5 during deployment or retrieval. The insertion/retrieval catheter 14 will further comprise a terminus end 18, slightly bent downward from the guide wire shaft 16. The locking cleat is a slot milled into the end to accept the small T anchor of the wall stent. The locking cleat engages the extraction point 7 of the spring stent 5, by inserting the small T anchor into the milled slot in the locking cleat of the catheter, during insertion or retrieval. When acted on by the user, the locking cleat puts a force on the extraction point 17, causing the spring stent to wind inward or outward as needed, thus shrinking or expanding the spring stent 5. This allows the spring stent to be inserted into the vessel or retrieved from the vessel.

For insertion into a vessel, the procedure is done under X-ray guidance in a sterile fluoroscopy procedure room, an initial angiogram and intravascular ultrasound are performed to confirm diagnosis and areas of vein narrowing, the proper diameter is selected using an intravascular ultrasound measurement which is already commonly used (IVUS). The flexible spring stent 5 is compressed at manufacturing onto a delivery flexible catheter 14 with metal hollow core that can be advanced over a commonly used vascular guide wire 16 (typically 0.035 inch). The spring stent catheter 14 is inserted over the guide wire 16 into the area of vein compression or narrowing under X-ray guidance. The catheter terminus end 18 is made with a small cleat that captures and holds the spring stent 5 extraction point 7. Once the spring stent 5 is in position, the catheter 14 and locking cleat can be turned clockwise over the wire 16 to allow the spring stent 5 to start expanding to its inherent manufactured diameter. The spring stent 5 is selected such as its diameter is 1-2 mm larger than normal vein diameter, so once expanded, it is seemed to the vein wall. The spring stent 5 is inherently self-expandable to resist outside compression in such a way to keep the root vein open and widened as close as possible to its natural diameter and surface area to allow normal return venous flow.

Once the deployment position is verified, the locking cleat can be disengaged from the extraction point 7, and the delivery catheter 14 and wire then removed. Before disengagement, if adjustment is needed, the spring stent 5 can be rotated counterclockwise, by handle 6 ad collar 8, to decrease the diameter of the spring stent 5 to match the delivery catheter 14, and the catheter 14 can be moved over the wire in either position, central or peripheral to the needed position before final delivery using the aforementioned described technique.

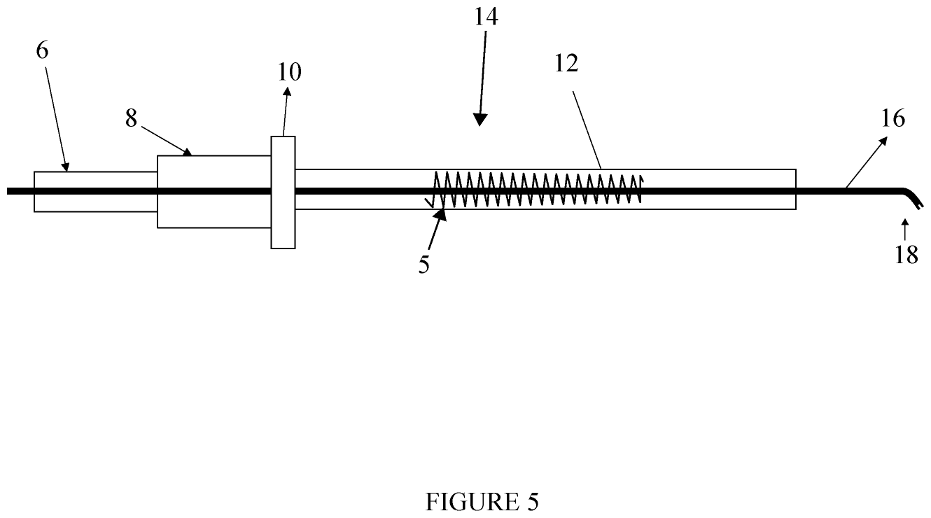

Conversely, for retrieval, the guide wire 16 is inserted to the location of the spring stent 5 under X-ray guidance, intravascular ultrasound device can be used for additional visualization from inside of the vessel to the precise tilt of anchor. The insertion/retrieval catheter 14 is deployed over a guide wire to the location of the spring stent's extraction point 7. The locking cleat engages the extraction point 7 and, while holding the collar mount 8, the handle 6 is turned counterclockwise to compress the spring stent 5 onto catheter 14. Once fully compressed, a long sheath 12 can be advanced over the catheter and the spring stent for removal as shown in FIG. 5.

A spring die could be used to create the spring stent 5. The speed of the spring die would control the diameter of the spring stent. The spring stent 5 could be formed in an operating room or it could be manufactured and sent prepackaged in the catheter based on desired predetermined multiple lengths and diameters. While there are obvious advantages to creating the spring stent 5 in the operating room (control the length to suit individual patients) it may be desirable to have them prepackaged as it would not also require know-how to create the spring stent. The spring stent 5 will compress within a catheter by elongating. Once the compression site is reached the spring stent 5 will be deployed and regain its spring shape.

By deploying the spring stent to the root vessel compression area, narrowing location or locations, the compression of the inferior vena cava is reduced or eliminated therefore allowing the blood to more easily flow back to the patient's heart. This increased flow will reduce or eliminate the pooling blood. Without the pooling blood, the patient will see a reduction in chronic symptoms of venous flow congestion that lead to many organs dysfunction, such as varicose veins, healing of ulcers, along with benefits to other chronic illnesses.

In another embodiment of the invention, the spring stent could be used to alleviate L4L5-S1 lumbar pain. The inventor believes that blood pools at the intersection of the aorta and the inferior vena cava. This is due to the restriction caused by the aorta crossing over the inferior vena cava. As the blood flow is restricted there, the blood seeks the path of least resistance. Given that veins have a porous quality when congested, the blood will seep out of the vein and pool in and around the L4L5-S1 vertebra. The blood that pools around the L4L5-S1 vertebra has been deoxygenated and contains toxins and blood free radicals from the body's organs. The toxin laden, deoxygenated blood will, over time, begin to break down the cells and cartilage of the vertebra and in much the same way it causes leg ulcers, it will deteriorate the vertebra causing pain and discomfort in the patient. By decreasing or removing the compression of the inferior vena cava through the use of a spring stent, the blood pooling around the L4L5-S1 vertebra can be reduced or eliminated, alleviating the L4L5-S1 lumbar pain.

The toxin laden, deoxygenated blood contains CO₂, toxic metabolites and free radicals from the body's organs. This combination creates a toxic environment for human cells and can break them down over time. The use of a spring stent will reduce or stop blood pooling in the patient's leg and, overtime, cure the ulcer and prevent further ulcers from occurring by treating the cause of the vein disease.

In yet another embodiment of the preferred invention the spring stent could be used to treat heart and arterial diseases such as hypertension. It is theorized that the body is over-compensating for a lack of return blood flow by increasing the pressure of the blood pumping into the arteries. This increased pressure results in hypertension. Therefore, if the return blood venous flow is restored, it is believed that the heart and body would then reduce the pressure on the arteries and therefore reduce or eliminate hypertension.

In yet another embodiment of the invention, the spring stent could be manufactured into a Y shape (not shown) so that it could treat both the right and left iliac veins simultaneously without require two separate deployments. This embodiment would prevent a single stent or two single stents from slipping or moving within the interior vena cava. This can also be used in patents where there is compression in both the right and left iliac vein. This Y shaped spring stent would also be retrievable and be deployed in a similar method to that of the spring stent.

The spring stent can be used to treat any chronic illness associated with the pooling of toxin laden, deoxygenated blood in undesirable locations, including but not limited to, chest paints, hypertension and hardening of arteries, blood clots in lungs known as deep venous thrombosis DVT/PE, chronic headaches, gastrointestinal diseases such as gall bladder dysfunction, irritable bowel syndrome, inflammatory bowel disease, undiagnosed abdominal pain, constipation, lower back pain, fibromyalgia, GYN disease such as endometriosis and chronic pelvic pain, neurodegenerative disease such as dementia (sundowning), psychiatric illnesses such as ADHD, autism, anxiety, depression, chronic narcotic addiction, immunity disease, such as allergies and asthma, obstetrics disease such as intra uterine growth restriction, leg swelling, varicose veins, postpartum depression, placenta congestion, bleeding during pregnancy, miscarriages, eclampsia, and preeclampsia, hypertension, miscarriages, eclampsia, preeclampsia, Musculoskeletal disease such as failed back syndrome, disc herniation, osteoarthritis, sacroiliitis, and disc deterioration not related to injury. Others such as restlessness leg syndrome, poor concentration, migraines and sciatica

We claim:

1. A retrievable stent comprising made of shape memory alloy:
a distal end and a proximal end in communication with one another, forming a helical shape;
the distal end terminating into an extraction point;
the proximal end terminating into an extraction point;
wherein the retrievable stent decreases in size from the distal end to the proximal end; and
wherein the shape memory alloy is self-expanding and capable of being collapsed.

2. The retrievable stent according to claim 1, wherein the extraction point is an eyelet.

3. The retrievable stent according to claim 1, wherein the extraction point is a T shaped anchor.

4. A retrievable stent comprising a proximal end and a distal end in communication with one another wherein the proximal end and the distal end form a helical shape;
a larger distal end and a smaller proximal end;
an extraction point comprising an eyelet;
wherein the frequency of the helical shape increases at the proximal end and at the distal end and decreases at the proximal end and distal end;
wherein the eyelet is capable of receiving a locking cleat from a catheter such that when force is applied to the extraction point the helical shape will expand or contract; and
wherein the retrieval stent may be removed by applying force to the eyelet via the catheter locking via the locking cleat to collapse the catheter and remove it from its position.

5. A retrievable stent comprising made of shape memory alloy:
a distal end and a proximal end in communication with one another, forming a helical shape;
at least one extraction point;
wherein the retrievable stent decreases in size from the distal end to the proximal end; and
wherein the shape memory alloy is self-expanding and capable of being collapsed.

6. The retrievable stent according to claim 5, wherein the shape metal alloy is Inconel.

7. The retrievable stent according to claim 5, wherein the at least one extraction point is in communication with the proximal end.

8. The retrievable stent according to claim 5, wherein the at least one extraction point is in communication with the distal end.

9. A retrievable stent comprising:
a distal end and a proximal end in communication with one another, forming a helical shape;
at least one extraction point; and
wherein, upon deployment, the retrievable stent expands and conforms to the size of the vessel.

10. The retrievable stent according to claim 9, wherein the diameter of retrievable stent is decreased when force is applied to the extraction point by an extraction catheter.

11. The retrievable stent according to claim 9, wherein the stent is made from a shape memory alloy.

12. The retrievable stent according to claim 9, wherein the retrievable stent decreases in size from the distal end to the proximal end

13. The retrievable stent according to claim 9, wherein the pitch of the helical shape is increased and the proximal and the distal ends.

14. A helical shaped retrievable stent comprising:
a distal end and a proximal end in communication with one another and forming the helical shape;
at least one extraction point in communication with at least one of the distal end or proximal end;
wherein, the helical shaped retrievable stent is made from a shape memory alloy capable of being expanded and contracted; and
upon deployment, the retrievable stent expands to conform to the size of the vessel.
